(19) United States
(12) Patent Application Publication
(10) Pub. No.: US 2002/0013955 A1
(43) Pub. Date: Jan. 31, 2002

(54) PRODUCTION OF RECOMBINANT PROTEIN IN TRANSGENIC FISH

(76) Inventors: SHARON OGDEN, ALAHAU, FL (US); SHELDON M. SCHUSTER, GAINESVILLE, FL (US)

Correspondence Address: MARTIN L MCGREGOR, MCGREGOR AND ALDER, 26415 OAK RIDGE, SPRING, TX, 77380

(21) Appl. No.: 09/095,192

(22) Filed: Jun. 10, 1998

(51) Int. Cl. A01K 67/00; A01K 67/033; A01K 67/027; C12P 21/00

(52) U.S. Cl. 800/20; 800/4

(57) ABSTRACT

This invention is a transgenic fish that expresses an amino acid sequence (either peptide or protein) under control of a chemical substance when the chemical substance is supplied to the fish. The protein will preferably be a heterologous protein, such as a protein useful as a pharmaceutical product in humans, or animals. The chemical substance may be a hormone or hormone mimic, such as a steroid, thyroid, retinoid and vitamin D. Especially preferred are fish responsive to estrogens and having estrogen responsive elements in the regulatory sequences for a heterologous protein. The transgenic fish may express a desired heterologous protein in a specific tissue such as a particular organ, especially preferred fish expresses a heterologous protein or peptide in the liver. Another preferred fish expresses a protein or peptide in the egg.

Alternatively this invention may be viewed as a method for production of a desired amino acid sequence comprising the steps of producing a construct of a DNA sequence comprising a DNA sequence coding for a desired amino acid sequence; inserting the DNA sequence coding for the desired protein into the genome of a fish such that the expression of the DNA sequence coding for the desired amino acid sequence is under the control of a regulatory region of DNA that regulates the expression of the amino acid sequence in response to a chemical substance, when the chemical substance is supplied to the fish. In another embodiment this invention is a method of producing a desired amino acid sequence in a fish comprising providing a chemical substance to a transgenic fish having a gene for expression of the desired protein under control of a regulatory element in the transgenic fish that regulates production of the desired protein in response to the presence or absence of the chemical substance. Preferred chemical substances are hormones or hormone like molecules such as steroids, thyroid hormones, retinoids and the D vitamins.

### TECHNICAL FIELD

This invention relates to the use of transgenic fish as a host system for the production of recombinant peptides and proteins. More specifically it concerns the expression of the recombinant protein in the organs of fish using expression vectors containing transcriptional elements derived from genes that are highly expressed in fish liver. In particular the invention teaches the use of gene regulatory elements derived from the fish genome that are inducible by the exogenous application of estrogen or related compounds.

### BACKGROUND OF THE INVENTION

Production of recombinant proteins has been accomplished in several different systems, including bacteria, yeast, baculovirus-infected insect cells, mammalian cells in culture, plant cells and in the organs of transgenic animals. Production of recombinant proteins in prokaryotic and lower eukaryotic organisms is limited by the inability of these systems to properly fold and/or post-translationally modify the proteins. Improperly folded proteins are often biologically inactive or alternatively, may not be useful as clinical agents because they do not survive in the circulation of the treated individuals into whom they are introduced. Examples of post-translational modifications of proteins which can affect either activity or lifetime in the circulation include phosphorylation, acetylation, amidation, vitamin K-dependent γ-carboxylation and glycosylation. Proteins which require such modifications, e.g., the blood clotting factors normally made by the liver, currently are made as recombinant DNA-expressed products either in mammalian cells in culture, or alternatively, in the organs of transgenic animals.

Expression levels of such modified proteins in mammalian cells in culture is typically low even under optimized growth conditions and from genes under the control of active regulatory elements such as promoters and enhancers. The advent of gene introduction techniques that allow transgenic animals to be developed from gene constructs delivered into fertilized eggs has enabled the expression of a protein in selected organs of animals harboring the introduced gene. These transgenic animals provide an alternative expression method for complex proteins. International Patent Application WO 98/15627 discloses transgenic fish expressing heterologous proteins in tissue and eggs but makes no reference to organ specific production nor of control by a chemical substance.

It has been shown that levels can be improved 10-1000 fold over those obtained in culture by expressing the genes for the proteins in the mammary organs of transgenic animals. The higher expression of proteins in whole organs within animals has been attributed to the 10-100 fold greater density of cells in organs versus cells in culture. Other factors for the increased production of proteins within organs have been suggested. These include the necessity for cells to be aggregated or to properly form the basement membrane as exposed in an organ in order to express proteins at high levels.

Transgenic non-human animals bearing an activated oncogene were claimed in U.S. Pat. No. 4,736,866. The technique disclosed teaches the construction of a plasmid that is injected into the male pronucleus of a single-cell, fertilized mouse egg and implanted into pseudopregnant female mice. The offspring were cross-bred to produce homozygous transgenic mice. A substantial number of such transgenic animals are known to exist including cattle with human lactoferrin genes and the like. Transgenic salmon having an antifreeze gene promoter to increase growth hormone production are disclosed in U.S. Pat. No. 5,545,808. Isolation of the gene for insulin-like growth factor from rainbow trout is disclosed in U.S. Pat. No. 5,476,779.

To date the focus for protein production in transgenic animals for commercial purposes, has been on the expression of transgenes in the mammary glands of large agricultural animals such as goats, pigs, sheep, and cattle. Research studies for transgenic protein expression frequently use the mouse as a model system that does not lend itself to commercial protein production. Although recombinant protein production levels can be high, the large animals are limited by several factors, including their long gestational period, small litter size, limited availability of fertilized eggs and technical difficulties in the micro injection techniques used to introduce DNA into the fertilized egg. Regulated expression of recombinant proteins in cells in culture has been described U.S. Pat. No. 5,534,418 using systems in culture which include specifically either a glucocorticoid receptor, mineralocorticoid receptor, thyroid receptor or estrogen-related receptor (ERR) and their cognate ligands, but use of this technique in whole animals has not been disclosed. Many of these limitations may be circumvented by using an alternate transgenic host such a transgenic fish. In order to develop a commercially feasible transgenic strain it would be desirable to rapidly evaluate a number of expression vector constructs in the host animal. The short gestation period and the availability of a large number of eggs will enable one skilled in the art to select the desirable expression vector that may be used to create the transgenic organism.

### SUMMARY OF THE INVENTION

This invention is a transgenic fish that expresses an amino acid sequence (either peptide or protein) under control of a chemical substance when the chemical substance is supplied to the fish. The peptide or protein will preferably be a heterologous peptide or protein, such as a peptide or protein useful as a pharmaceutical product in humans, or animals. The chemical substance may be a hormone or hormone mimic, such as a steroid, thyroid hormone, retinoid or a D vitamin. Especially preferred are fish responsive to estrogens and having estrogen responsive elements in the regulatory sequences for a heterologous protein. The transgenic fish may express a desired heterologous protein in a specific tissue such as a particular organ, especially preferred fish expresses a heterologous protein or peptide in the liver. Another preferred fish expresses a protein or peptide in the egg.

Alternatively this invention may be viewed as a method for production of a desired amino acid sequence comprising the steps of producing a construct comprising a DNA sequence coding for a desired amino acid sequence; inserting the DNA sequence coding for the desired protein into the genome of a fish such that the expression of the DNA sequence coding for the desired amino acid sequence is under the control of a regulatory region of DNA that regulates the expression of the amino acid sequence in response to a chemical substance, when the chemical substance is supplied to the fish. In another embodiment this invention is a method of producing a desired amino acid sequence in a fish comprising providing a chemical substance to a transgenic fish having a gene for expression of the desired protein under control of a regulatory element in the transgenic fish that regulates production of the desired protein in response to the presence or absence of the chemical substance. Preferred chemical substances are hormones or hormone like molecules such as steroids, thyroid hormones, retinoids and the D vitamins.

Preferred regulatory sequences include vitellogenin promoters, choriogenin H promoters, and Choriogenin L promoters. Preferred control sequences are estrogen response elements selected from the group consisting of estrogen response elements, tamoxifen response elements; and androgen response elements. In another embodiment the invention provides a method of protein production which comprises constructing a transgenic fish that expresses a desired heterologous protein and isolating the protein from the fish in quantities in excess of 1 mg/g of tissue weight. The invention in another aspect provides a transgenic fish that expresses a heterologous protein in quantities greater than 1 mg/g of tissue weight. The invention also provides in its broadest aspect a method for controlling production of a heterologous amino acid sequence in a specific organ of a transgenic animal which comprises introducing into the genome of the animal a construct which comprises a DNA sequence coding for the amino acid sequence to be expressed operably linked to a chemical substance responsive gene expression regulatory sequence such that the amino acid sequence is expressed in response to exogenous application of a hormone in a target organ.

### DETAILED DESCRIPTION OF THE INVENTION

### Definitions

“Vector” means a not naturally occurring DNA construct that contains a sequence coding for a specific amino acid sequence in operable linkage with other DNA sequences that promote the expression of the sequence and control expression of the amino acid sequence. Optionally a vector may also include elements that induce the propagation of the vector in a host cell.

“Transgenic Animal” means a whole multicellular organism, such as a fish, having a genome containing a vector.

“Fish” as used here means an egg laying member of the classes Agnathia, Chondrichthyes, and Osteichthyes. Preferred are bony fish of Class Osteichthyes.

“Receptor” means a cellular moiety that can specifically bind to a particular small class of non-homologous molecules with a high degree of specificity and affinity. The affinity being at least eight orders of magnitude greater than the affinity of the moiety for most other naturally occurring molecules.

“Ligand” as used here means a molecule that specifically binds to a particular Receptor.

“Estrogen” means a steroid or synthetic polycyclic molecule having the ability to bind to and activate an Estrogen Receptor. Preferred natural estrogens include estrone (Δ^{1,3,5}-estratrieneol-3-one-17), α-estradiol (Δ^{1,3,5}-estratrienediol-3,17), and estriol (Δ^{1,3,5}-estratrienetriol-3,16,17) and 17β estradiol (Δ^{1,3,5[10]}-estratriene-3,17β-diol). Preferred synthetic estrogens are stilbestrol, (3,4-di(4-hydroxyphenyl)hex-3-ene), hexestrol, benzestrol and dienestrol.

“Estrogen Response Elements” (‘ERE’) means a DNA sequence that binds an estrogen receptor (ER) and when an estrogen is bound to the ER, the ER-estrogen-ERE complex changes the transcription of a proximate DNA sequence coding for an amino acid sequence. An ERE may be included in a Vector.

“PCR” means Polymerase Chain Reaction as taught in Mullis, U.S. Pat. No. 4,683,195.

“Promoter” means a DNA sequence controlling expression of a gene or DNA sequence encoding an amino acid sequence. Normally a promoter will include a binding site for an RNA polymerase to initiate transcription

“Transcription” means the process of copying a strand of DNA to yield a complementary strand of RNA.

“DNA” means Deoxyribonucleic Acid.

“cDNA” means a complementary DNA sequence obtained by reverse transcription of mRNA.

“RNA” means Ribonucleic Acid.

“mRNA” means messenger RNA.

“Antibody” means a product of an immune system cell having a characteristic heavy and light chain structure and specifically binding a particular antigen.

The production of transgenic fish has thus far been limited to two areas: the development of species with improved performance traits and the use in the study of development, as in the patents listed above. The development of transgenic fish for the purposes of high level protein production in specific organs under control of a supplied chemical substance has heretofore not been described. The focus of this application, the production of transgenic fish expressing large quantities of recombinant protein for purification and commercial use, is therefore novel. The production of protein in transgenic fish has several advantages over methods employing other transgenic hosts (Gong and Hew, Curr. Topics Dev. Biol., 30: 177-214 (1995)). One individual fish provides from 100's to 1000's of eggs, so that the availability of eggs for DNA transfer is not limiting. Fertilization is external, allowing easy collection of freshly fertilized eggs. Some species of fish, such as tilapia sp., produce eggs every 2-3 weeks, allowing large numbers of fish and gametes to be produced in a very short period of time, DNA can be introduced into the fertilized eggs by any conventional technique for introduction of DNA into a cell such as microinjection, the DNA gun, transfection or electroporation. The preferred method is electroporation, a technique in which the eggs are bathed in a solution of the DNA to be incorporated and an electric current is applied which opens pores in the eggs long enough for surrounding DNA to enter the egg. The use of this technique requires no special skill on the part of the investigator, and hundreds of potential transgenic fish can be created within a single experiment (Inoue et al., Cell. Differ. Dev., 29: 123-129 (1990)). The ability to use this technique to create transgenic animals offers significant advantages over the micro injection techniques currently employed to produce transgenic mammals. After fertilization, fish develop without further manipulation and can be tested for the presence of a transgene within a week after hatching, using standard methods. The short generation time for several species of fish also offers an advantage over large mammalian species.

### Expression of Genes in Fish

In order to express a recombinant protein from DNA in a living system, it is necessary to include sequences which direct the transcription of the gene, the gene sequence encoding the protein, and sequences which direct the termination of transcription of the gene. Sequences which direct the transcription of a gene are generally regions located adjacent to the 5′ end of a gene and are termed “promoters”. Transcription termination signals are located beyond the 3′ end of the coding region of the gene and often contain sequences AATAAA followed by stretches of variable length comprising pyrimidine rich sequences. Genes to be expressed may be derived from cDNAs produced from mRNA isolated from any biological source expressing the gene of interest, or alternatively, may be derived from genomic DNA isolated from the species of interest. Genomic DNAs are often very large, containing not only the sequence for the gene, but a variable number of introns (intervening sequences removed from the RNA transcribed from a gene) as well. There may be some advantage to including introns in genes to be expressed in transgenic animals.

Many promoters have been used in attempts to produce transgenic fish with economically desired characteristics, including those isolated from mouse leukemia virus (MoMLV), mouse κ immunoglobulin gene, SV40 virus, promoters containing the Rous sarcoma virus long terminal repeat (RSV LTR), mouse metallothienin gene, Xenopus laevis elongation factor 1α gene, carp β-actin gene, human heat shock protein gene, ocean pout antifreeze protein gene and the VSV promoter (Fletcher and Davies, Genetic Engineering 13: 331-370 (1991); Liu et al., Bio/Technology 8: 1268-1272 (1990); Du et al., Biotechnology 10: 176-181 (1992)). All have yielded limited expression in species of fish studied (goldfish, loach carp, rainbow trout, Atlantic salmon); none except carp β-actin promoter and ocean pout antifreeze promoter, are native to fish. It is expected that promoter regions isolated from fish will be optimally expressed in a fish system, since the regulatory regions are expected to optimally bind to regulatory proteins residing in the homologous species.

Since to date transgenic fish have been made mainly for the purpose of enhancing fish performance and not to serve as host for production of heterologous proteins for the purpose of manufacturing recombinant proteins, it has not been necessary to use very highly expressed, regulated gene expression. High levels of expression of exogenous proteins are likely to be detrimental to the development and maintenance of the fish, and it is therefore desirable to include within the transgene elements which will allow the gene to be highly expressed and also to be regulated as desired. Thus far no such system has been described in the production of transgenic fish and therefore, the description of such a system, according to the present application, is novel.

### Regulated Gene Expression in Higher Eukaryotic Cells

Transcriptional regulation of gene expression is temporally and spatially regulated by a wide variety of complex and interconnecting mechanisms. Amongst the best studied regulatory systems are those in which the control of gene expression is regulated by a superfamily of nuclear receptors, including receptors for hormone or hormone like molecules such as steroid, thyroid hormone, retinoid and vitamin D. Regulation of gene expression by hormone receptors occurs via the following general mechanism: Receptors specific for each hormone reside within the cell. When the cognate hormone is present, it binds to the receptors and causes an activation of the receptor, such that the receptor-hormone complex is capable of binding to specific DNA sequences within the regulatory regions of hormone-responsive genes. These DNA elements, termed hormone response elements (HREs), act as enhancer elements to stimulate transcription in response to the presence of the hormone. Regulated expression of recombinant proteins in cells in culture has been described U.S. Pat. No. 5,534,418 using systems in culture which include specifically either a glucocorticoid receptor, mineralocorticoid receptor, thyroid receptor or estrogen-related receptor (ERR) and their cognate ligands, as noted above. Estrogen-related receptors are members of the nuclear receptor superfamily by sequence homology, but the ligands and functions for this group of receptors are unknown. Estrogen, for example, is not a ligand for ERR function. The mechanisms of binding to DNA and the DNA sequences to which they bind distinguish them from the classical nuclear hormone receptors. The present application is distinct from these regulatory systems. The regulation of recombinant gene expression using the exogenous application of a hormone in concert with hormone-regulated recombinant genes within organs of whole animals have heretofore not been described. More specifically the use of such systems to create transgenic fish suitable to produce recombinant proteins is new.

### Hormone Regulation of Vitellogenin Gene Expression in a Fish

Vitellogenins (VTG) are egg yolk proteins made in livers of oviparous animals at the time eggs are formed, i.e. at sexual maturity. Several genes for VTGs are found in all species which have been examined. VTG production in female liver is under the stringent control of estrogens, and can be made in either males or females of oviparous species in response to the exogenous administration of estrogen or other estrogenic compounds which can work as estrogen receptor agonists. Estrogen induces very high levels of VTG gene expression in the liver: it has been estimated that 50% of the mRNA produced after estrogen stimulation is from the vitellogenin genes. Other egg yolk proteins are made at the same time under control of estrogen or agonists. Choriogenin H and Choriogenin L are egg envelope proteins found in teleosts, which are liver proteins regulated by estrogen. Apo VLDL II, another estrogen-regulated liver protein, is a lipid transporter characterized primarily in chickens. Any of these genes contain regulatory elements that can be transposed to a recombinant gene to effect inducible regulation by estrogen or related agonists.

The molecular mechanisms whereby estrogen specifically regulates gene expression have been extensively studied. Estrogen regulates specific gene expression by binding to a receptor (estrogen receptor, ER); the receptor then undergoes a series of measurable changes which are characteristic of an activated receptor. Activated receptors can transactivate expression of genes when bound to specific sequences within those genes called estrogen response elements (EREs). EREs can be found in the promoter region, or in the transcribed region of genes. Within the transcribed region, EREs can be located within the protein coding sequence, within the introns, or within the untranslated regions of the gene. All function to confer control by estrogens via binding of estrogen or agonist to the ER, which then is activated and binds to the ERE. Other estrogen regulated proteins which bind to specific regions of estrogen-controlled genes can be used to regulate transcription. Such proteins have been identified for the 3′ untranslated region of the X. laevis VTG gene. The level of ER in a cell is thought to be rate-limiting for estrogen-regulated gene expression. ER gene expression is autoregulated; increasing the number of ER genes or increasing the level of expression of existing genes is likely to increase expression from genes positively regulated through EREs. Sequences encoding binding regions for accessory proteins can augment or modify ERE function providing the accessory protein is present. Thus increasing the expression of ERs or accessory proteins within transgenic animals is a method by which regulated gene expression can be augmented.

### Vector Construction

Vectors for the expression of recombinant proteins in transgenic fish contain the following elements: a promoter region containing DNA sequences which confer estrogen-inducibility in the livers of the transgenic fish, the complete transgene to be expressed derived from an isolated cDNA or genomic clone containing introns, a 3′-untranslated sequence which either does not contain instability elements or alternatively, contains elements which are stabilized in the presence of estrogen (Dodson and Shapiro, Mol. Cell. Biol., 14: 3130-3138 (1994)), and sequences which allow the propagation in bacterial host vectors. The native promoter for a vitellogenin gene congenic with the transgenic host is desirable because it is likely to express the transgene at the highest levels. For expression of recombinant protein in tilapia this promoter can be cloned from isolated genomic DNA using standard methods including the polymerase chain reaction (PCR).

Genomic DNA is isolated by homogenizing the liver, ovary or other organ from one adult tilapia in an extraction buffer containing 50 mM NaCi, 10 mM Tris-HCl, pH 8.0, 0.1 M EDTA, pH 8.0, 0.5% SDS. Once tissue is homogenized, protease K is added to a final concentration of 100 μg/ml and the solution is stirred and incubated at 37° C. for 1-16 hr. An equal volume of phenol pre-equilibrated with 0.1 M Tris-HCl, 10 mM EDTA is added, mixed by inversion and the subsequently centrifuged at 5000 g for 15 min. to separate the phases. The aqueous phase is re-extracted with phenol until no protein is visible at the interphase and the resulting DNA is then dialyzed against a solution of 20 mM Tris-HCl, 1 mM EDTA, pH 8.0 to remove the phenol.

To PCR clone the tilapia vitellogenin promoter the following primers, derived from sequences obtained from GENBANK (accession #Z71336), are used:

5′CATTCAGCATTGCTGAGCATC

3′GCAGAGGCGTCCTTTTTAAGC

Alternatively, any set of primers from this sequence which include the putative EREs may be used.

The PCR reactions (50 μl) are carried out using a commercially available kit (e.g. 1578 553 from Boehringer Mannheim), by combining in a thin-walled microtiter tube the following reagents:

5 μl 10×PCR buffer (from kit)

1 μl 5′ primer (10 μM)

1 μl 3′ primer (10 μM)

0.5 μl Taq DNA polymerase (from kit)

0.1 μl dNTP (from kit)

36.5 μl H₂O

5.0 μl boiled genomic DNA

Amplification is carried out in a thermocycler, using a step-down program which anneals the primers at a temperature which decreases from 60° to 47° C. at a rate of 0.2°/sec at each cycle. A 34 cycle program is used. After the PCR program is complete, 3.0 μl of 0.5 M EDTA is added to each reaction and 10 μl is removed for visualization using standard gel electophoresis procedures. A 1.6 kb band is expected using the 2 primer sequences given. This promoter fragment may be cloned into any appropriate vector which contains as elements a reporter gene or other transgene in which the gene product may be assayed as well as 3′ sequences which will direct the termination of transcription. Standard methods are used and standard vectors are available e.g., the pNASSβ vector from Clontech. Alternatively, the PCR product can be cloned into a conventional T/A cloning vector available from Clontech or other vendors, excised with a variety of restriction enzymes and subsequently ligated into any expression vector using conventional methods.

Expression vectors contain transcriptional termination sequences including polyA addition sites and introns to increase gene expression. Those transcription termination regions which can be found in commercially available vectors include those derived from SV40 early polyA addition element, SV40 late polyA addition element, or human growth hormone. In addition, expression vectors contain a reporter gene or multiple cloning sites in which can be inserted any gene for which a clone is available. Genomic clones and cDNA clones are commonly modified so that they do not contain 3′ untranslated sequences before insertion into these vectors. Reporter genes which are currently available include β-galactosidase, chloramphenicol acetyl transferase (CAT), luciferase and green flourescence protein. Vectors containing this vitellogenin promoter are expected to confer on the gene inserted just 3′ of the promoter both liver specific and estrogen-regulated expression, as is the case for the endogenous vitellogenin gene.

### Modified Vitellogenin Promoter-containing Vectors—Enhanced Expression with Altered EREs

The construction of vectors which predictably will respond to estrogen and related agonists and antagonists differentially with respect to the endogenous gene may be desirable so that the administration of subphysiological levels of estrogen can be used to induce expression of the transgene, without co-inducing expression of the endogenous estrogen-regulated genes. Expression of the recombinant protein and purification of this protein from transgenic fish liver are expected to be optimized if, for example, endogenous vitellogenin expression is not co-induced. It is predicted that since the presence of multiple, tandemly arranged EREs can function synergistically, the native VTG gene promoter can be modified to be more sensitive to estrogen by inserting extra EREs into the promoter as described in Anolik et al., Biochemistry 34: 2511-2520 (1995).

The sequences of functional EREs have been determined from several genes in many different species. These sequences resemble the canonical sequence first described from the X laevis vitellogenin gene: GGTCAnnnTGACC. Many functional EREs are palindromic, but many contain changes in either the 5′ half or the 3′ half so that they are no longer palindromic. Sequences can also function as EREs if only one half of the palindromic sequence, or a variation of it, is located at variable distances from other half-sites. The ERE sequence, spacing of half-sites, surrounding sequences and presence of other activator sequences all contribute to the estrogen-regulated expression of the gene, but identification of a sequence as a functional ERE usually requires that specific criteria be met.

EREs in published sequences may be recognized according to the following criteria:

1. Relation to canonical sequence

2. In vitro binding to ER regardless of presence of agonist or antagonist. Binding is specific, i.e., can be competed by other sequences containing EREs, but not by non-ERE containing DNA.

3. Bending of DNA containing putative ERE when ER is bound and differential bending when ER is bound to agonist versus antagonist.

4. When ligated in cis to reporter gene, confer estrogen or agonist inducibility when transfected into cells in culture or into organisms or oocytes or fertilized eggs or developmental stages of animals, provided that these living systems contain functional estrogen receptors. Estrogen receptors may also be absent from cells and can be supplied via cotransfection of expression plasmids containing the genes for the estrogen receptor.

ERE's can be excised from genomic DNA or can be synthesized according to standard procedures. These sequences can be inserted within or adjacent to the native promoter using conventional procedures. For example, unique Xba I, Nco I, Hinc II and Pvu II restriction sites exist in the tilapia vitellogenin promoter fragment cloned using the primers discussed previously. EREs are synthesized with the ends flanked by sequences containing the restriction sites, then ligated into these sites using standard procedures. Clones containing one or more of the inserted sequences are identified by PCR analysis of individual bacterial colonies. Such vectors are expected to to display increased and synergistic responses to the addition of estrogenic compounds (Mattick et al., J. Steroid Biochem. Molec. Biol 60: 285-2940 (1997)).

A priori it is not always possible to predict which modified constructs will confer enhanced estrogen inducibility. Therefore, it is desirable to use convenient assays in cells prior to testing the constructs in transgenic animals.

### Heterologous Control

Promoters conferring estrogen regulation on a transgene can be further modified so that the transgene expression may be independently controlled relative to the expression of endogenous vitellogenin and other egg proteins. This would be desirable, for example, when the transgenic females are used to produce transgenic offspring. Secondary regulation systems can be used to activate or repress transcription in response to specific inducers. A preferred system comprises a promoter and/or gene segment containing a binding sequence for a protein not normally found in the host, a gene encoding the regulatory protein and a specific chemical inducer or co-inducer or repressor or co-repressor which binds to the regulatory protein, causing it either to bind to the specific regulatory sequence, or alternatively, to cause a bound protein to be released from the specific regulatory sequence. The regulatory protein binding sequence may then be inserted at the 5′ end of the gene or at any other sequence position such that when the regulatory protein is bound to the sequence, the gene is prevented from being transcribed. Examples of such control systems include the tetracycline binding protein from E. coli, its regulatory sequence and tetracycline, and the arabinose binding regulatory protein from E. coli (araC), its regulatory sequences and arabinose or fucose or other inducers/repressors. Many other systems with these kinds of components would function as well. Such regulatory systems would allow the production of a transgene to be repressed in the presence of estrogen. It is expected that reproductive functions of the fish would proceed normally, in spite of presence of estrogen regulation of the inserted transgene.

### Transient Expression Assays to Test Constructs

In order to test the functionality of constructs before they are introduced into fertilized eggs of fish, a transient expression assay system comprised of a DNA containing an assayable reporter transgene linked to the promoter to be tested, and a cell line which can be transiently transfected and which contains elements necessary for promoter activation is used. For example, in order to test the functionality of an estrogen-inducible promoter construct, such as the constructs containing vitellogenin or related promoters, the cell line used as the host for the transient transfection assay must contain estrogen receptors. Alternatively, DNAs containing the coding regions for the estrogen receptor under control of a strong constitutive promoter such as the CMV immediate early promoter, can be cotransfected with the DNA to be tested. It is desirable that the transient assay system be as closely related to the transgenic host as possible, so that the results of such assays may be used as a basis for selection of expression vector constructs in the transgenic host.

A preferred system is one in which the cells are uniform in function with regard to promoter activation. A continuous cell line derived from the organ of or closely related to the transgenic host is preferred, but other cells containing the necessary transactivating cellular machinery can also be used. For example, MCF-7 cells, a human breast cancer line which contains functional estrogen receptors, can be used as the host for transient assays of estrogen-inducible promoters

One preferred assay system comprises a continuous fish liver cell line e.g., RTH149 cells as the expression host and transient transfection of plasmids containing promoters to be assayed. Such cells will contain liver-specific signal transduction elements and are preferred hosts for evaluating expression levels from expression vector constructs subsequently used to produce transgenic fish. Another method is to isolate primary cultures of fish liver cells using standard methods for establishing primary liver cultures as the host for the transient assays (Flouriot et al., J. Cell Sci., 105: 407-416 (1993)). Another method is to use any estrogen responsive cell line, e.g., human breast cancer cells such as MCF-7 cells, for assay of estrogen responsiveness of promoter elements. The X laevis vitellogenin gene-derived ERE has been shown to be functional and responsive to estrogen in MCF-7 cells, suggesting that the use of this cell line provides a viable assay system for the evaluation of expression vector constructs.

Transient transfection assays are carried out using standard assay methods. These assays involve the incubation of tissue culture cells with the DNA to be assayed in the presence of reagents that facilitate the entry of DNA into the cells. In order to test for functional estrogen inducibility of genes the assays are carried out in the absence of estrogen, or alternatively in the presence of a known estrogen antagonist such as tamoxifen, as well as in several different concentrations of estrogen. A typical assay is performed as follows: cells are plated in microtiter 96-well tissue culture plates at a density of 5,000-10,000 cells per well. The tissue culture media used is dependent on the cell type, but typically contains a balanced salt solution such as DMEM along with fetal bovine serum which has been stripped of endogenous hormones by preincubation with charcoal and dextran (obtainable from HyClone, Inc) at a level of between 5-10% by volume. Typically, phenol red is left out of the media in these assays. Once cells are 50-80% confluent, DNA is added. One protocol for introducing DNA uses a cationic reagent, lipofectamine (Gibco-BRL) as the transfecting reagent. The lipofectamine reagent is diluted to 16 μl in a serum-free media or reduced-serum medium such as Opti-MEM (Gibco-BRL). Separately, the DNA to be transfected is diluted to 1 μg/ml in the same medium. The cells to be transfected are washed 3 times in a serum free media to remove serum and then equal volumes of diluted DNA and lipofectamine are added, followed by gentle mixing. Alternatively, the lipofectamine is premixed with the DNA and allowed to incubate for 15-45 min. prior to adding to the cells. Typically the final volume per well is 75-150 μl. Optimal levels of the transfecting reagent and DNA vary considerably and are determined for each cell type assayed. Cells are incubated with the DNA/lipofectamine mixture for 2-24 hrs., but typically 4-6 hrs is optimal. This incubation is usually carried out in a 37° CO₂ incubator. Following this incubation, the lipofectamine/DNA is removed and fresh media containing serum is added. Cells are further incubated for 24-72 hrs. before an assay is performed to quantify the expression of the transfected gene (reporter assay).

Reporter gene expression in these assays is enhanced if reporter gene expression driven by exogenous estrogen control sequences is carried out in the presence of co-transfected estrogen receptor. The preferred system contains fish estrogen receptor genes cloned into expression vectors so that high levels of transient expression of estrogen receptor are achieved. The sequence for the estrogen receptor from rainbow trout is known, and the gene may be cloned and inserted into expression vectors using known methods. Estrogen receptor genes from other species can be cloned by homology using known methods.

### Reporter Gene Assay for Transient Expression

Many different reporter genes can be used to test estrogen responsiveness of native and modified promoters. Specific kits and protocols are available from a variety of commercial sources. A reporter gene should encode a protein for which there is little or no activity in the cells or tissues to be assayed. One such reporter gene is chloramphenicol acetyl transferase (“CAT”), an enzyme found only in bacteria.

A CAT enzyme-linked immunosorbent assay (ELISA) kit is used to detect and quantify the presence of CAT protein in crude extracts of the transfected cells. This assay can be used at any time after the cells are transfected, but typically will be used only on cells that have been transfected for 24-72 hrs. at 37° C. Cells are frozen and thawed 3 times in 100 μl of 0.25 M Tris-Cl, pH 7.8. Cell extracts are then diluted 1:10 and 1:50, using a standard dilution buffer supplied in the ELISA kit, so that the concentration of CAT falls within the linear range of the assay (0.1-1.0 ng/ml). Polystyrene microwells supplied in the kit are supplied coated with a rabbit polyclonal antibody specific for the CAT protein. 200 μl of the cellular extract to be assayed is placed in each well. Incubation at room temperature for 2 hours allows any CAT present in the extract to bind to the antibody-coated surface. All of the unbound extract is then removed from the wells and the wells are washed 5 times with a standard washing buffer provided in the kit. 200 μl of a second polyclonal antibody which has been biotinylated and is also specific for CAT is then added to each well and allowed to bind to the CAT. Incubation is for 1 hr. at room temperature. The excess unbound biotinylated antibody is removed and the wells are washed 5 times to remove any unbound protein. 200 μl of alkaline phosphatase-conjugated to steptavidin is then added and allowed to bind to the biotin present in the CAT-antibody complex for 30 minutes at room temperature. Unbound protein is removed by washing the wells 5 times as described in previous steps. The amount of alkaline phosphatase is measured colorimetrically by adding to the wells 200 μl of a substrate solution containing 2 mg/ml para-nitrophenylphosphate dissolved in a diethanolamine buffer supplied with the kit. After 30 min. at room temperature, the alkaline phosphatase cleaves the phosphate from the substrate, leaving a yellow colored para-nitrophenol product which can be measured as an absorbance at 405 nm in a spectrophotomoter, or alternatively, by transferring the contents of each well to a microtiter 96-well plate and reading the absorbance of each well with a plate reader, e.g., using a Model 3550 plate reader from BioRad. A standard curve is prepared by diluting a known concentration of purified CAT (supplied in the kit) and performing the same assay simultaneously.

Using this assay on transfected cells that have been grown in the presence of different concentrations of estrogen or related compounds, it is possible to assess the estrogen-inducibility of many different constructs and to compare the relative sensitivity of modified promoters to the native promoter. It is predicted that the relative sensitivities of the constructs to estrogen in these assays will be reflected in the relative sensitivities of the constructs within the transgenic fish into which the constructs are introduced. These assays are also used to test heterologous control elements which have been introduced into some of the constructs, by performing the assays in the absence and presence of the heterologous regulatory reagents e.g., arabinose or tetracycline.

### Production of Transgenic Fish

While any fish species may be used according to the invention, for the production of amino acid sequences, teleost fish are preferred. Tilapia (Oreochromis aureas), zebrafish (Danio rerio), carp (Cyprinus carpto), Atlantic Salmon (Salmo salar), and rainbow trout (Orcorhyncuu mykiss) are especially preferred. It is convenient to use fish for which propagation and transgenic technology is well developed. Propagation is illustrated below for Tilapia.

A typical brood fish holding system consists of three 140 cm×84 cm×46 cm (440 liter) rectangular polyethylene or glass tanks. Each tank is equipped with a 600 liter per hour, rainbow lifeguard, fluidized bed biological filter. The filter contains a nitrification bacterial culture suitable for conversion of ammonia (NH₃) from fish waste to nitrate. A typical culture includes nitrosomonas strains that convert ammonia to nitrite (NO₂) and nitrobacter that convert nitrite to nitrate (NO₃). This biological process is performed in an aerobic environment. Dissolved oxygen levels are maintained in the range of 5 to 8 parts per million (ppm) using a pressurized air pump and two 2.5×5 cm porous airstones, preferably bonded silica airstones. The tanks are maintained at 26-31° C. by use of two 300 watt electric quartz heaters. Stocking densities are ten to fifteen, 450-500 g fish per tank. Fish are fed a suitable diet, for example Tilapia are fed a prepared diet of trout and catfish pellets. Feeds are manufacturer by Silvercup Feeds of Murray, Utah and are formulated to provide maximum growth and yolk production.

### Tilapia Propagation

Tilapia (Oreochromis aureas) are obtained from a local supplier and are maintained in 25 gallon aquariums in dechlorinated tap water at 26-31° C. To induce spawning, male and female breeding pairs are transferred to a separate tank maintained at 28-30° C. with a broad spectrum light regulated by a timer set to 14 hrs.on/10 hrs off.

Brood fish are individually tagged with color coded, streamer tags, secured to the first dorsal spine. Ripe females (possessing mature eggs) and males (with flowing milt) are injected with 0.5 to 1.0 ml of Ovaprim per kg of body weight. Injections are given IM, at the posterior junction of the dorsal fin and body. Ovaprim injections are divided into two injections, 8 hours apart. The first injection is 10% of the total, the second is the remaining 90% of the total. Females are monitored post injection for signs of ovulation by gentle palpitation. Female fish with free flowing eggs are removed from the tank, hand dried with a cloth to remove any water from their exterior, and the eggs are then hand stripped into a sterile bowl. A male fish with flowing sperm is removed from the tank, hand dried, and the milt hand-stripped into the bowl containing the eggs. Milt and eggs are gently mixed for 0.5 -1.0 min. Water from hatching containers is added to the bowl in a quantity that will just cover the egg and milt mixture, and again gently stirred for 1.0 to 2.0 minutes. Female fish produce mature eggs on an approximately thirty day cycle.

The fertilized eggs are put into an electroporation cuvette in a solution of calcium-free phosphate buffered saline. DNA to be electroporated is purified using standard methods, including banding in CsCl gradients 2 times. The DNA is linearized with an appropriate restriction enzyme that does not cleave within the promoter-transgene-terminator sequence insert and is cleaned by extraction with phenol, followed by ethanol precipitation. It is generally thought that linearized DNA is more likely to be integrated into the chromosome than supercoiled plasmid although the supercoiled form may also be used. DNA is resuspended to a final concentration of about 100 μg/ml and is used at this concentration for the electroporation. 0.8 ml of DNA solution is used together with 100-200 fertilized eggs in a 0.4 cm gene pulser cuvette (Bio-Rad) for each experiment. The DNA may be reused several times. Using a gene pulser plus apparatus (Bio-Rad), a typical electroporation of fish eggs is performed at a field strength of 100-250 V/cm, using a 0.25 μF capacitor. Typically 3 pulses are used with a time constant of 5-10 msec and a pulse interval of 1 sec, but other combinations of field strength and pulse generation may also be used. The electroporated eggs are transferred to the hatching system. Optimum electroporation results require careful optimization of electroporation protocols, and results tend to be instrument specific.

A typical hatching system consists of three 80 1 glass aquariums, each equipped with a 1000 L per hour magnetic drive submerged pump and a McDonald type hatching jar. Tanks are maintained at saturation using a 2.5×5 cm airstone and a pressurized air pump. Eggs are hatched by gently rolling them in the inverted bell hatching jar, using water from the aquarium, injected by the magnetic drive pump, through a center tube to the bottom of the hatching jar. Incubation time at 27° C. is 7-10 days. Swim up fry are allowed to overflow, from the hatching jar, into the aquarium. The newly hatched fry are fed, for the first 7 days, a diet of newly hatched brine shrimp. After 7 days the fry are weaned onto starter sized Silvercup trout diet.

### Assay for Transgene

Fin clippings from batches of 10 fry are screened via PCR analysis, using primers specific for the transgene. It is expected that the incorporation of the transgene will vary and that many batches of fry will not have transgenic members. DNA from the fins is isolated using standard techniques. Basically the small pieces of fin tissue (less then 1 cm in diameter from each fish) are cut, pooled in batches of around 10 clips per batch, pulverized in liquid nitrogen and then dissolved in a solution of guanidine thiocyanate, 0.3 M NaOac, pH 8.0. Proteins are removed by phenol extraction and the DNA is recovered by precipitation with ethanol. Positive batches are individually analyzed using an additional fin clip and a repeated PCR analysis.

### Propagation of Transgenic Fish

Once transgenic fish founders have been identified, further generations of transgenic fish can be obtained by cross-breeding transgenic males with normal females. It is expected that 50% of such offspring will be heterozygous for the transgene, although in the first generation it is expected that the number of transgenic offspring may be lower due to mosaicism associated with production of transgenic fish. Breeding of the fish is done by standard methods of temperature and light control. Once fry hatch, the presence of the transgene is determined by obtaining individual fin clips, preparing genomic DNA and performing PCR analysis using standard methodology.

### Production of Equine Chorionic Gonadotropin (eCG) in Transgenic Fish

Equine chorionic gonadotropin is an important target for commercial development because it is a potent inducer of follicle development in domestic and laboratory animals. To date it has been available only as a purified hormone derived from pregnant mare serum, an expensive and labor intensive procedure. The protein is highly glycosylated and cannot be made at present as a recombinant product in existing expression systems.

Equine CG consists of 2 subunits, α and β, which are the product of two genes expressed in the pituitary of horses. In the pituitary these two subunits make up luteinizing hormone, a glycoprotein hormone which is identical in gene structure to eCG derived from the placenta. The sequences of the genes for the α and β subunits of eCG are available from Genbank (Accession AB000200 and S41704). RNA is isolated from horse pituitary glands using a kit obtained from Qiagen, Inc. (Santa Clarita, Calif.). Basically, the tissue is homogenized in a solution supplied in the kit. The homogenate is then passed through a silica-gel membrane to which all RNA sticks. The RNA is eluted with a small volume of water and ethanol precipitated using well known procedures. Genomic DNA is purified using a similar kit from Qiagen. Either nucleic acid preparation may be used to obtain clones for the α and β subunits of eCG. Oligonucleotide primers are identified in the published sequences which are used to amplify the gene coding sequence. For example, to amplify the gene sequence encoding the a subunit, the following primers are used:

5′AGGAGAGCTATGGATTACTA

3′CACTTGGTGAAACCTTTAAA

To amplify the gene sequence encoding the β subunit, the following set of primers are used:

5′TAAACACGGCAGAGGAGGCA

3′TCAAGAAGTCTTTATTGGGAG

The PCR reactions (50 μl) are carried out using genomic DNA and a commercially available kit (e.g. 1578 553 from Boehringer Mannheim), by combining in a thin-walled microtiter tube the following reagents:

5 μl 10×PCR buffer (from kit)

1 μl 5′ primer (10 μM)

1 μl 3′ primer (10 μM)

0.5 μl Taq DNA polymerase (from kit)

0.1 μl dNTP (from kit)

36.5 μl H₂O

Amplification is carried out in a thermocycler, using a step-down program which anneals the primers at a temperature which decreases from 60° to 47° C. at a rate of 0.2°/sec at each cycle. A 34 cycle program is used. After the PCR program is complete, 3.0 μl of 0.5 M EDTA is added to each reaction and 10 μl is removed for visualization using standard gel electophoresis procedures. The DNA is cloned into the expression vectors containing the vitellogenin promoter and other regulatory elements using well known standard techniques.

### Induction of Production of eCG in Transgenic Fish Liver

Induction of transgene expression in the livers of transgenic fish is accomplished by exposing the fish to estrogen or related compounds via injection. Injections are i.p. with estrogenic compounds generally at 2-4 mg/kg. Typically 17β-estradiol is preferred, but other compounds like those described in VanderKuur et al., Biochemistry, 32: 7016-7021 (1993), can also be used. At times ranging from 2-24 hrs. post-induction, livers are removed from the fish and protein is purified.

### Purification of eCG

All steps of the procedure are done at 4 deg C. Livers are removed and homogenized in a small volume of 0.5 M NaOAc, pH 6.0 (1 ml/g liver) using a polytron homogenizer. 10 volumes of ethanol are added and the homogenate is centrifuged at 6000×g (4 deg C.) as described in Bousefield and Ward, J. Biol. Chem., 259: 1911-1921 (1984). Further extractions are performed as detailed for the isolation of lutropin from horse pituitary. Basically extractions are done sequentially in a series of buffers of decreasing ethanol contents. The second extraction is performed by resuspending the pellet from the first step in 20 volumes of 75% ethanol, 25% 0.5 M sodium acetate, pH 6.0, 2 hrs. The suspension is then spun at 6000×g , the pellet resuspended and extracted in 20 volumes of 60% ethanol, 40% 0.5 M sodium acetate, pH 6.0, 2 hrs, 4 deg C. After spinning, gonadotropin is extracted by resuspension of the pellet in 50% ethanol/1 M NaCl/0.5 M Tris-acetate, pH 7.0 followed by overnight incubation. The gonadotropin is centrifuged at 6000×g and the pellet is resuspended in 0.126 M ammonium bicarbonate and dialyzed against 0.005 M sodium phosphate, pH 6.0 overnight. The resulting dialyzed material is applied by batch loading to CM-sephadex that has been prewashed with all of the eluting buffers. After binding for 2 hrs, unbound material is removed by washing the column material with 5 column bed volumes of 0.01 M sodium phosphate, pH 6.0, followed by washing in 5 column bed volumes of 0.04 M sodium borate, pH 8.3. After removing all of the excess wash solutions the slurry of CM-sephadex bound to equine gonadotropin is puored into a column and allowed to settle. The bound protein is then eluted with 0.2 M NaCl/0.04 M sodium borate pH 8.3. The partially purified eCG is dialyzed against 1.0 mM sodium phospate, pH 6.8, and applied to a hydroxyapatite column. As described in Moore and Ward, J. Biol. Chem., 255:6923-6929 (1980), it is important that the loaded protein be at a certain concentration (4.3-7.5 mg/ml hydroxyapatite) to maximize yields of active protein. Active protein does not bind to the column, and is found in the flow-through. Purity of the final material is determined using standard methods of SDS gel electrophoresis and amino acid sequence analysis.

We claim:

1. A transgenic fish that expresses a peptide or a protein under control of a chemical substance when the chemical substance is supplied to the fish.

2. A transgenic fish according to claim 1 that expresses a heterologous peptide or protein.

3. A transgenic fish according to claim 1 that expresses a peptide or a protein in response to an exogenous chemical substance.

4. A transgenic fish according to claim 1 that expresses a peptide or a protein in response to a chemical substance selected from the group consisting of steroids, thyroid hormones, retinoids and D vitamins.

5. A transgenic fish according to claim 1 having an estrogen responsive element regulatory sequence controlling expression of a peptide or a protein.

6. A transgenic fish according to claim 1 having a regulatory sequence selected from the group consisting of vitellogenin promoters, choriogenin H promoters, and Choriogenin L promoters.

7. A transgenic fish according to claim 1 having a control sequence selected from the group consisting of estrogen response elements, tamoxifen response elements; and androgen response elements.

8. A transgenic fish according to claim 1 that expresses a peptide or a protein in a particular organ.

9. A transgenic fish according to claim 1 that expresses a peptide or a protein in liver.

10. A method for production of a desired amino acid sequence in a transgenic fish comprising the steps of producing a construct comprising a DNA sequence coding for a desired amino acid sequence; inserting the construct into the genome of a fish such that the expression of the DNA sequence coding for the desired protein is under the control of a regulatory region of DNA that regulates the expression of the protein in response to a chemical substance, when the chemical substance is supplied to the fish.

11. A method according to claim 10 wherein the fish expresses a heterologous amino acid sequence.

12. A method according to claim 10 wherein the fish expresses an amino acid sequence in response to a hormone or hormone mimic.

13. A method according to claim 10 wherein the fish expresses a protein in response to a chemical substance selected from the group consisting of steroids, thyroid hormones, retinoids and D vitamins.

14. A method according to claim 10 wherein the construct comprises an estrogen responsive element regulatory sequence controlling expression of an amino acid sequence.

15. A method according to claim 10 wherein the construct comprises a regulatory sequence selected from the group consisting of vitellogenin promoters, choriogenin H promoters, and Choriogenin L promoters.

16. A method according to claim 10 wherein the fish expresses an amino acid sequence in a specific tissue.

17. A method according to claim 10 wherein the fish expresses an amino acid sequence in a particular organ.

18. A method according to claim 10 wherein the fish expresses an amino acid sequence in liver.

19. A method according to claim 10 wherein the fish expresses a heterologous protein in the liver.

20. A method according to claim 10 wherein the fish expresses an amino acid sequence in the egg.


SEQUENCE LISTING (6 sequences)

SEQ ID NO: 1 - DNA, 18 bp, Artificial Sequence
  Description of Artificial Sequence Primer for PCR
cattcagcattgagcatc

SEQ ID NO: 2 - DNA, 21 bp, Artificial Sequence
  Description of Artificial Sequence Primer for PCR
gcagaggcgtcctttttaagc

SEQ ID NO: 3 - DNA, 20 bp, Artificial Sequence
  Description of Artificial Sequence Primer for PCR
aggagagctatggattacta

SEQ ID NO: 4 - DNA, 20 bp, Artificial Sequence
  Description of Artificial Sequence Primer for PCR
cacttggtgaaacctttaaa

SEQ ID NO: 5 - DNA, 20 bp, Artificial Sequence
  Description of Artificial Sequence Primer for PCR
taaacacggcagaggaggca

SEQ ID NO: 6 - DNA, 21 bp, Artificial Sequence
  Description of Artificial Sequence Primer for PCR
tcaagaagtctttattgggag